(19) United States
(12) Plant Patent Application Publication
(10) Pub. No.: US 2005/0022281 P1
(43) Pub. Date: Jan. 27, 2005

(54) Zoysiagrass plant named 'Z-89'

(50) ZOYSIA SP.; ZOYSIAGRASS

(76) Inventor: John W. B. Rockie, Kaneohe, HI (US)

Correspondence Address: CHRISTIE, PARKER & HALE, LLP, PO BOX 7068, PASADENA, CA, 91109-7068, US

(21) Appl. No.: 10/624,966

(22) Filed: Jul. 21, 2003

(51) Int. Cl. A01H 5/00

(52) U.S. Cl. PLT/390

(57) ABSTRACT

Described is a new Zoysiagrass variety characterized by a finer leaf texture, an improved color and an improved growth habit compared to other Zoysiagrass varieties.

### LATIN NAME OF GENUS AND SPECIES

The present invention relates to a new Zoysia sp. plant.

### BACKGROUND OF THE INVENTION

The present invention relates to a new and distinct variety of Zoysia sp., which is a member of the Graminae family.

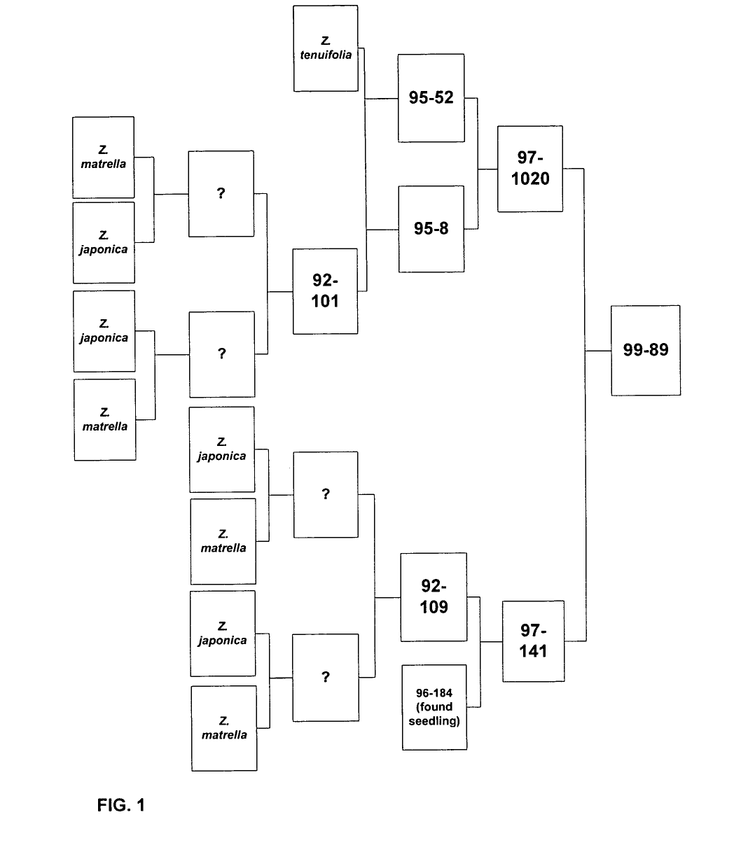

The new variety, Z-89, also referred to herein as Z99-89, is the result of five generations of crossing, beginning with a cross of Z. japonica×Z. matrella, and the reciprocal cross; Z. tenuifolia was introduced in the third generation of crosses. The pedigree is shown in FIG. 1. The first cross was field-pollinated. All succeeding generations of crosses were hand-pollinated under greenhouse conditions, and seed was germinated under greenhouse conditions. Seedlings were individually cultured in a greenhouse before being transplanted to the field for evaluation and selection.

Periodic drought conditions in Hawaii have increased the need for improved warm season turfgrass varieties. Zoysiagrasses are well adapted for use under such conditions because of their low water use rates and high heat tolerance.

### SUMMARY OF THE INVENTION

The new variety of zoysiagrass, Z-89, exhibits a finer leaf texture, an improved color and an improved growth habit compared to other currently available commercial zoysiagrass varieties. The new variety is vegetatively propagated from sod, plugs, tillers or stolon and rhizome pieces. Asexual propagation in Oahu, HI has established that the combination of chartacteristics of the new variety are transmitted through succeeding generations. However, seed is genetically unstable.

### BRIEF DESCRIPTION OF ILLUSTRATION(S)

FIG. 1 is a diagram showing the pedigree of Z-89.

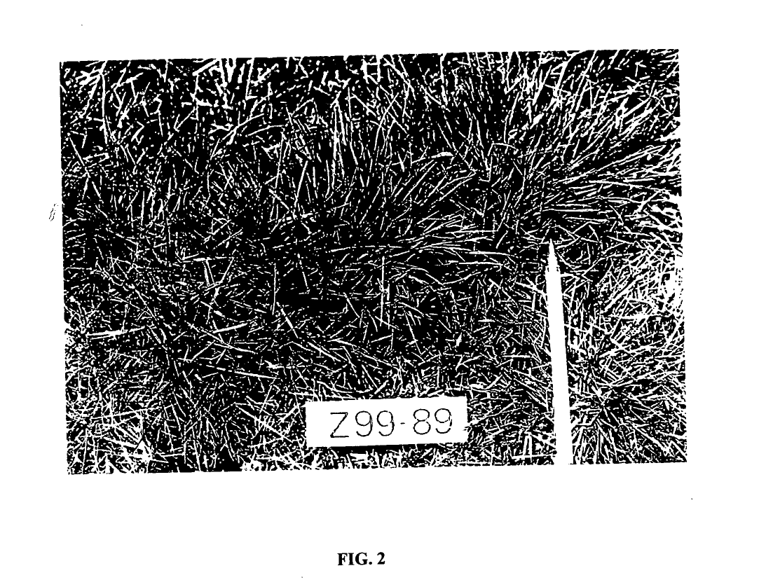

FIG. 2 shows the turf appearance of the new variety.

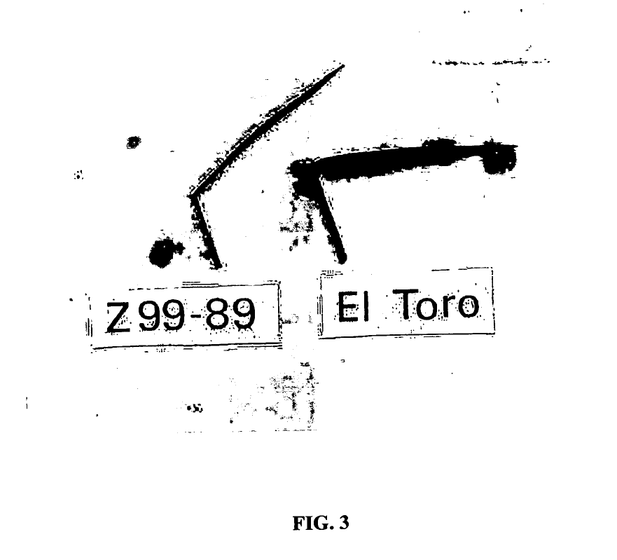

FIG. 3 shows the appearance of a single leaf of the new variety as compared to that of ‘El Toro.’

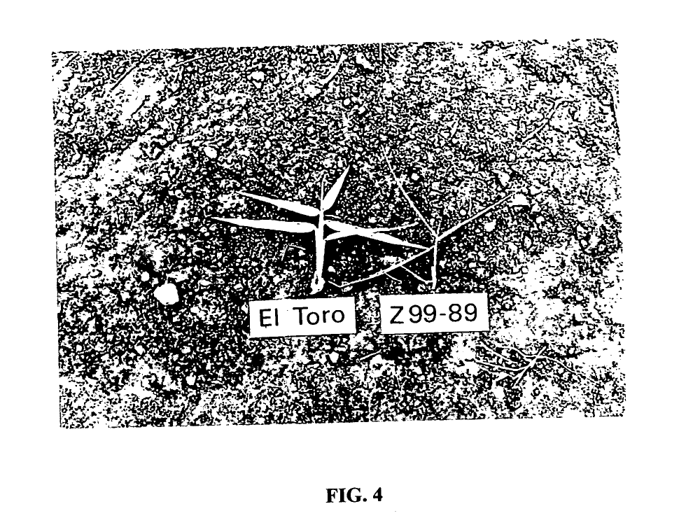

FIG. 4 depicts the leaf orientation of the new variety and of the ‘El Toro’ variety.

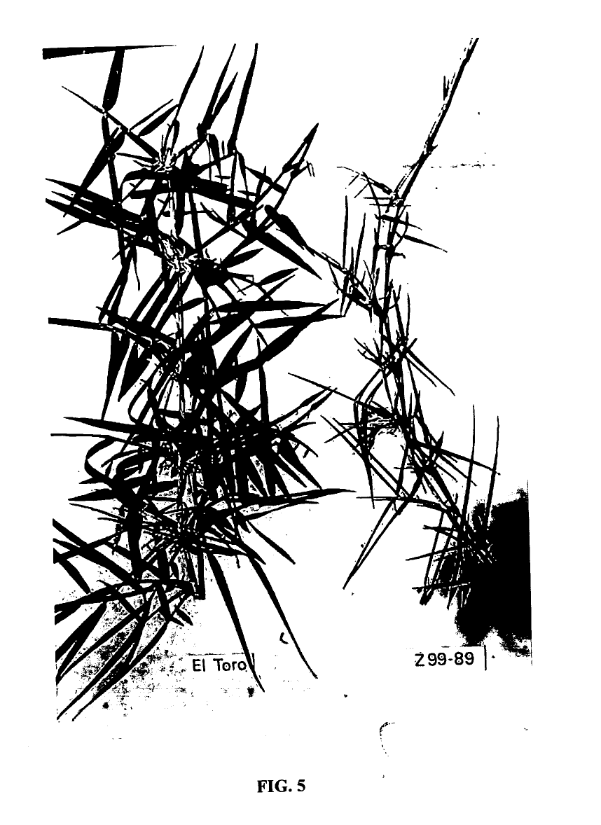

FIG. 5 shows stolons of the new variety compared with ‘El Toro.’

### DESCRIPTION OF THE NEW VARIETY

The new variety ‘Z-89’ is characterized by a finer leaf texture, an improved color and an improved growth habit compared to other currently available commercial Zoysiagrass varieties. The following description is of 8 months-old plants grown in Oahu, HI with color descriptions in accordance with the Color Chart of The Royal Horticultural Society, London (4^{th} ed. 1995).

All dimensions are in millimeters, weights in grams (unless otherwise stated).
Leaf color.—Near 137A (upper surface); near 139A (underside).
Stolon color.—Near N79B.
Leaf length.—From 3.2 cm to 9.9 cm; average length 5.7 cm.
Leaf width.—From 1.5 mm to 3.0 mm; average width 2.2 mm.
Internode length.—From 0.4 cm to 2.9 cm; average length 1.7 cm.
Internode thickness.—From 1.2 mm to 2.1 mm; average 1.6 mm.
Raceme length.—From 2.7 cm to 9.9 cm; average length 5.0 cm.
Culm height.—From 7.5 cm to 13.1 cm; average 9.0 cm.
Pedicel.—From 1.0 mm to 2.1 mm; average 1.5 mm.
Ligule.—Hairs at ligule area from 2.5 mm to 3.0 mm; hairs on side of ligule 0.5 mm to 4.0 mm.
Spikelet with anthers and stigma exerted.—Color from near N77C to near N186C.
Mature spikelets.—Color from near N79A to near 158A.

Winter color retention and spring green-up are impossible to evaluate in Hawaii's climate.

1. A new Zoysiagrass plant of the variety substantially as shown and described.
